(19) United States
(12) Patent Application Publication
(10) Pub. No.: US 2025/0213131 A1
(43) Pub. Date: Jul. 3, 2025

(54) INDUCTANCE PLETHYSMOGRAPHY DEVICE

(71) Applicant: ETISENSE, LYON (FR)

(72) Inventors: Timothé FLENET, LYON (FR); Charles EYNARD, LYON (FR); Corentin BOUILLON, LYON (FR)

(21) Appl. No.: 18/873,985

(22) PCT Filed: May 10, 2023
(86) PCT No.: PCT/EP2023/062480

§ 371 Date: Dec. 11, 2024

(30) Foreign Application Priority Data
Jun. 13, 2022 (FR) 2205661

(51) Int. Cl. A61B 5/0535; A61B 5/00; A61B 5/113

(52) U.S. Cl. CPC A61B 5/0535; A61B 5/1135; A61B 5/6823; A61B 5/6831; A61B 2503/40; A61B 2562/12; A61B 2562/227

(57) ABSTRACT

The invention relates to an inductance plethysmography device comprising: a support (20) made of elastic textile designed to be closed on itself along two lateral edges (21, 22) in order to surround a portion of the torso of an individual, at least one inductive coil (80) formed of an electrically conductive wire strand having two ends arranged on each lateral edge of the support, characterized in that a first end of the coil (80) comprises a first electrical connection element (41) electrically and mechanically connected to an electrically conductive male or female self-gripping element (51), and a second end of the coil (80) comprises a second electrical connection element (42) electrically and mechanically connected to an electrically conductive female or male self-gripping element (52) designed to engage with the electrically conductive male or female self-gripping element in order to make an electrical contact between the first and the second end of the coil (80).

### FIELD OF THE INVENTION

The present invention relates to an inductance plethysmography device and a method for manufacturing such a device.

### STATE OF THE ART

Inductance plethysmography is a tool for measuring volume variations in the trunk of a mammal (human or animal), particularly used for respiratory and/or cardiac monitoring applications. The volume of the trunk is reconstructed using one or more sensors positioned to measure variations in mechanical compartments considered independent (generally the abdomen and the thorax).

The principle of this measurement is to wrap around the subject's trunk at least one inductive coil formed of one or more electrically conductive wire strands. A variation in the volume of the trunk, due in particular to respiratory and/or cardiac activity, generates a variation in the inductance of each coil connected to the surface of the contour surrounded by said coil, the variation in inductance being measured by a sensor.

Typically, such a device is integrated into a textile support such as a vest or a band to be worn on the body in order to ensure its retention on the subject. The support must be both extensible and of sufficient elasticity in order to ensure a permanent mechanical coupling between the band and the portion of the subject's trunk which is surrounded in such a way that any variation in the section of the subject's trunk generates an equivalent variation in the surface surrounded by the coil. The mechanical stress (in particular pressure and rigidity) imposed on the subject wearing the textile support must be minimal so as not to disturb the measured physiological phenomena and not to induce discomfort. The behavior of the subject, in particular attempts to remove such a textile support and/or manipulations out of curiosity of the subject wearing the garment or other individuals, for example fellows in the case of a plurality of animals kept together must be taken into account. Thus, for animals and young children and more generally any subject who is not able to consent to wearing the vest, the support must be designed in such a way as to prevent and protect against interactions with other animals (pulling, biting, attempted removal, etc.) and to not be able to be removed without the intervention of an operator.

Document FR 3094628 A1 discloses a closed tube-shaped inductance plethysmography vest for a small mammal. Such a vest is suitable only for small mammals weighing less than 6 kg, such as lagomorphs. For reasons related to the morphology, the greater force and the more complex handling of the subjects, it is difficult to put such a vest on the trunk of a larger animal or of a different species, and to keep it in place.

Furthermore, such a vest is not suitable for subjects undergoing a large increase or decrease in the perimeter of the trunk as is the case for example for young animals in the growth phase and/or in the case of significant weight gain or loss induced by a pathology. In the case of rapid weight gain, the vest can compress the subject, which causes discomfort and can lead to injuries and attempts to destroy and/or remove the vest as well as a disruption of the physiological phenomena measured. Conversely, in the case of weight loss, the vest is not adjusted to the trunk and the poor mechanical coupling does not allow reliable measurements. Since the elasticity of the vest is limited by both the textile support and the conductive wire used for their production, a multitude of sizes will be necessary in the case of repeated monitoring on an individual with a large increase or decrease in the perimeter of the trunk.

### DISCLOSURE OF THE INVENTION

A purpose of the invention is to provide an inductance plethysmography device allowing to adapt the size according to the weight of the individual wearing it even in the event of a significant increase or decrease in the perimeter of the trunk, for example for a young animal in the growth phase, or in the event of significant weight gain or loss due to a pathology.

For this purpose, the invention proposes an inductance plethysmography device comprising:
a support made of elastic textile designed to be closed on itself along two lateral edges in order to surround at least a portion of the trunk of an individual,
at least one inductive coil formed of an electrically conductive wire strand arranged in the form of wavelets between the two lateral edges so as to extend along the circumference of the individual's trunk, the coil having two ends arranged on each lateral edge of the support,
   characterized in that a first end of the coil comprises a first electrical connection element electrically connected to the electrically conductive wire strand, said first electrical connection element being electrically and mechanically connected to an electrically conductive male or female self-gripping element,
   and a second end of the coil comprises a second electrical connection element electrically connected to the electrically conductive wire strand, said second electrical connection element being electrically and mechanically connected to an electrically conductive female or male self-gripping element designed to electrically and mechanically engage with the electrically conductive male or female self-gripping element in order to make an electrical contact between the first and the second end of the coil.

Advantageously, each electrical connection element is a printed circuit board or an electrically conductive rigid plate.

Preferably, the device comprises a plurality of female or male self-gripping elements electrically and mechanically connected to the second electrical connection element, said female or male self-gripping elements being spaced apart by a distance between two successive self-gripping elements, such that each female or male self-gripping element defines a different circumference of the coil when said female or male self-gripping element is engaged with the male or female self-gripping element electrically and mechanically connected to the first electrical connection element.

The first electrical connection element and the second electrical connection element may each have a respective rigid base.

Each male or female self-gripping element can be assembled to the first electrical connection element or to the second electrical connection element by an electrically conductive glue.

Advantageously, each male or female self-gripping element is assembled to the first electrical connection element or to the second electrical connection element by at least one rivet. Said rivet may be electrically conductive.

In some embodiments each self-gripping element and each respective electrical connection element are arranged between two rigid plates connected by said rivet.

Preferably, each self-gripping element and/or the textile support is arranged between a rigid plate and the electrical connection element, the rigid plate and the electrical connection element being mechanically and electrically connected by said rivet.

Advantageously, the plethysmography device further comprises at least one pair of hooks arranged on each lateral edge of the support so as to mechanically connect said lateral edges to close the support.

In some embodiments, the device further comprises a removable elastic textile covering part arranged to cover the male and/or female self-gripping elements when the coil is closed.

Preferably, the textile support comprises at least one pair of hooks arranged on each lateral edge and the covering part includes at least one pair of loops arranged to be hooked onto said hooks so as to secure the covering part to the support and to cover the lateral edges of the support.

Advantageously, the covering part further comprises a pocket configured to contain a measuring device electrically connected to the coil.

The invention also relates to a method for manufacturing an inductance plethysmography device for a human or animal mammal as described above, comprising
providing the support made of elastic textile,
forming at least one inductive coil from an electrically conductive wire by securing said wire in the form of wavelets along the circumference of the support, said coil having two ends arranged on each lateral edge of the support,
assembling each end of the coil with a strip of the printed circuit board, so as to establish an electrical connection between the coil and a conductive track of the strip of the printed circuit board,
assembling each electrical connection element with a respective male or female self-gripping element.
   Preferably, the method further comprises assembling at least one electrical connection element with at least one additional self-gripping element, said self-gripping elements being spaced apart by a distance extending over a segment of the circumference of the device.

Preferably, the method comprises providing a covering part comprising a plurality of loops, and assembling the support with a plurality of hooks on both sides of the opening of said support, said loops being configured to receive the hooks so as to secure the covering part to the support.

### BRIEF DESCRIPTION OF THE FIGURES

Other features and advantages of the invention will emerge from the detailed description which follows, with reference to the appended drawings, in which:

FIG. 1 shows the outer face of an inductance plethysmography device according to the invention.

FIG. 2 shows the inner face of an inductance plethysmography device according to the invention.

FIG. 3 shows an inductance plethysmography device worn by an animal.

FIG. 4A and FIG. 4B illustrate the principle of closing each inductance loop.

FIG. 5 shows a printed circuit board providing electrical contact between the electrical wire and snap fasteners.

FIG. 6A and FIG. 6B illustrate the riveting of snap fasteners with a printed circuit board and a rigid plate.

FIG. 7A and FIG. 7B illustrate a first embodiment of riveting a gripping strip with a printed circuit board and a pair of rigid plates.

FIG. 7C and FIG. 7D illustrate a second embodiment of riveting a gripping strip with a printed circuit board on a rigid base and a rigid plate.

FIG. 7E illustrates the gluing of an elongated gripping strip.

FIG. 7F illustrates a set of gripping strips glued to a printed circuit board and a textile support.

FIG. 8 shows a covering part comprising a pocket for a power supply.

### DETAILED DESCRIPTION OF EMBODIMENTS

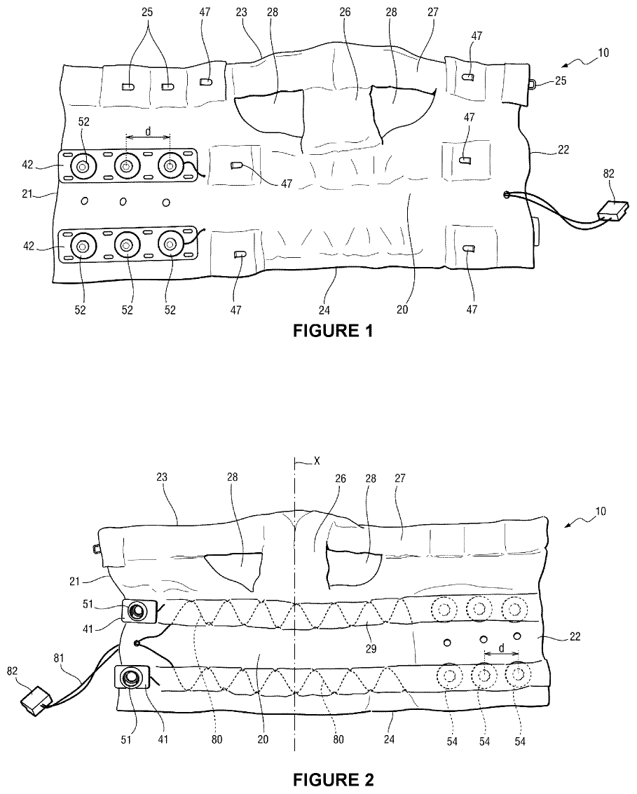

FIG. 1 shows the outer face of a plethysmography device 10 according to the invention. This device is intended to be worn by a mammal, which may be a human or an animal. In the remainder of the text, the term “individual” will generally refer to the subject wearing the device. The terms “front”, “anterior” or “upper” will be used interchangeably to designate an element located on the side of the individual's head (the latter may be a biped or a quadruped), and “rear”, “hind” or “lower” an element located on the opposite side.

The device comprises a support 20 made of elastic textile, for example a knitted textile or a fabric comprising fibers made of an elastic material such as elastane.

The size and shape of the support 20 are designed to the mammal species intended to wear the device 10. The elasticity of the support 20 ensures a certain comfort of wearing the device 10 and a good hold on the individual. It also allows to compensate within certain limits for differences in morphology and size between different individuals, or of the same individual whose morphology changes over time.

In some cases, supports 20 may be provided in different sizes to be selected based on the weight and circumference of the individual wearing the device 10.

### Textile Support

The textile support 20 typically comprises an upper edge 23 intended to surround the trunk near the neck of the individual wearing the plethysmography device 10, a lower edge 24 intended to surround the trunk of the individual closer to the hind or lower limbs, and two lateral edges 21, 22. The term “length” denotes the dimension of the support extending between the upper edge and the lower edge and the term “width” denotes the dimension extending between the two lateral edges.

The support 20 is designed to be closed on itself along said lateral edges 21, 22 in order to surround at least a portion of the individual's trunk. Consequently, when the support is placed on the individual, the length of the support extends substantially parallel to the individual's spine, and the width of the support extends along the circumference of the individual's trunk.

Typically, the support 20 surrounds an upper segment of the trunk, for example the torso. The support 20 may comprise opening and closing elements 25, such as pairs of hooks or hooks and loops. These opening and closing elements 25 may be arranged along the lateral edges 21, 22 or only on certain areas, for example on the outer face of the upper edge 23. This allows to secure the support on the trunk of the individual and possibly to prevent the individual from removing the support 20 for example by passing the head through the opening created by the upper edge 23 surrounding the neck.

Advantageously, the textile support 20 comprises openings 28 designed to pass the anterior or upper limbs of the individual intended to wear the device 10. The use of such openings 28 allows to hold the support in place and, in the case of an animal, to prevent the individual from removing it alone or with the help of fellow animals.

The support 20 may comprise a padded segment 26 between the openings 28 for placement in contact with the mammal's chest, and/or a padded segment 27 at the upper edge. Such padded segments 26, 27 increase comfort when the support 20 is worn, and stabilize the support 20 on the individual's trunk to hold it firmly in place without causing pressure points or pain.

In some embodiments, the support consists of a fabric strip without openings for limbs. Such an arrangement is often perceived as more comfortable by a subject consenting to the plethysmography examination, for example a human adult. It is also simpler to make.

### Inductive Coils

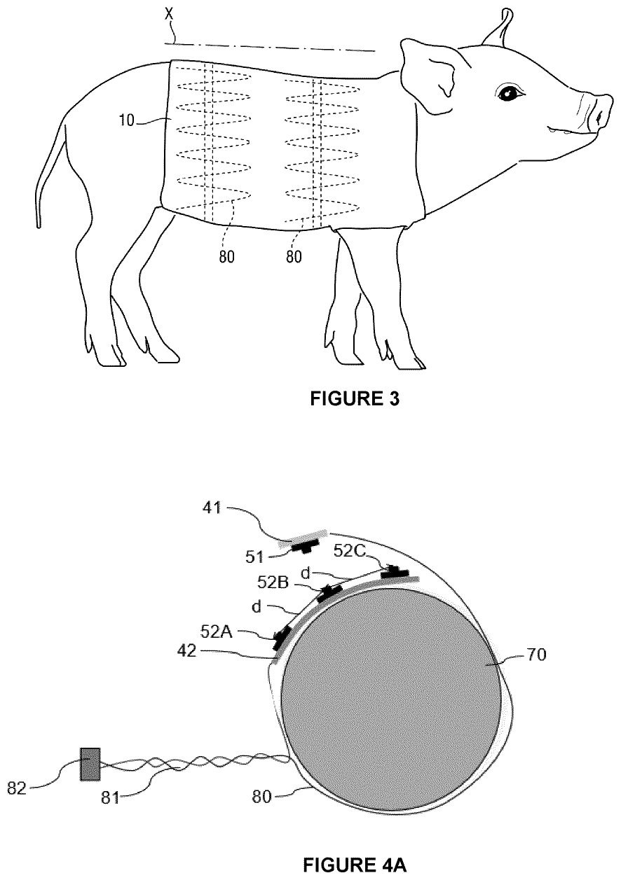

With reference to FIG. 3, the plethysmography device 10 further comprises at least one inductive coil 80 intended to surround the individual's trunk.

Each inductive coil 80 is formed of an electrically conductive wire strand 81, such as a copper wire, including electrical insulation. The conductive wire strand 81 is arranged in wavelets along the circumference of the textile support. With reference to FIG. 2 which shows the inner face of the device, the wire strand 81 is held in this shape for example by a textile tunnel 29 sewn onto the inner face of the support 20 and appropriate intermediate seams. The two ends of the conductive wire 81 are arranged near the respective lateral edges 21, 22 of the textile support 20. The wavelet shape adapts to variations in the diameter of the trunk surrounded by the support 20, and thus allows precise measurements of the surface surrounded by the coil 80. The coils 80 further comprise an electrical connector 82 arranged at the end of the electrically conductive wire strand 81. Such a connector 82 is designed to electrically and mechanically engage with a connector of a measuring device. All coils of a plethysmography device are connected to the measuring device to ensure synchronous recording of measurements.

The ends of the conductive wire strand not being arranged in wavelets and extending beyond the textile support are not intended to surround the individual's trunk. It is therefore sought to minimize the surface surrounded by these ends and to prevent any variation in the residual surface in this area to improve the accuracy of the measurements. For this purpose, the ends of the conductive wire 81 are twisted.

Preferably, the device comprises between 1 and 4 coils. The use of a plurality of coils 80 results in redundancy of measured data, thus allowing to increase the accuracy and reliability of the measurements.

Each inductive coil 80 includes at each of its ends an electrical connection element 41, 42. Typically, such an electrical connection element is a printed circuit board. The electrical connection element 41, 42 is electrically connected to the conductive wire strand, for example by soldering. Typically, the printed circuit board 41, 42 comprises a base of the printed circuit board, one or more electrical contact areas 45 allowing an electrical connection with a connection element 52 which will be described below, and at least one conductive track configured in order to make an electrical contact between the conductive wire strand 81 and the electrical contact area(s) 45. In a preferred embodiment, the entire surface of the printed circuit board is electrically conductive in order to obtain a low impedance and good resistance to possible twisting and/or shearing of the printed circuit board. Each base 41, 42 is held near the lateral edge 21 or 22 of the textile support 20 by one or more seams. The contact areas 45 and the conductive tracks or surfaces are printed on the bases of the printed circuit board 41, 42 made of an electrically conductive material.

Alternatively, the electrical connection element 41, 42 is an electrically conductive rigid plate, for example a metal plate.

Typically, a first end of each coil 80 arranged on a first lateral edge 21 of the textile support includes a printed circuit board 41 having a single electrical contact area 45. This first printed circuit board 41 is arranged on a preferably rigid base to facilitate the opening and closing of the connection elements which will be described below.

In some embodiments, a second end of each coil arranged on the second lateral edge 22 of the textile support 20 includes a printed circuit board 42 having a plurality of electrical contact areas 45. This second printed circuit board 42 is arranged on a flexible base to adapt to the shape of the individual's trunk when the device is worn. Typically, the base of the printed circuit board 42 arranged near the second lateral edge 22 is in the form of an elongated strip along an axis P perpendicular to the lateral edge 22 of the textile support 20 and tangential to the perimeter of the mammal's trunk when the plethysmography device 10 is worn. The electrical contact areas 45 are arranged along the axis P of the support and spaced apart by a distance d between centers of two adjacent contact areas 45.

In some embodiments, the printed circuit boards on the two ends of a coil 80 include several contact areas 45 along an axis P perpendicular to the respective lateral edge 21, 22 of the textile support 20. In this case, the two printed circuit boards 41, 42 include flexible bases capable of following the shape of the individual's trunk.

### Connection Elements

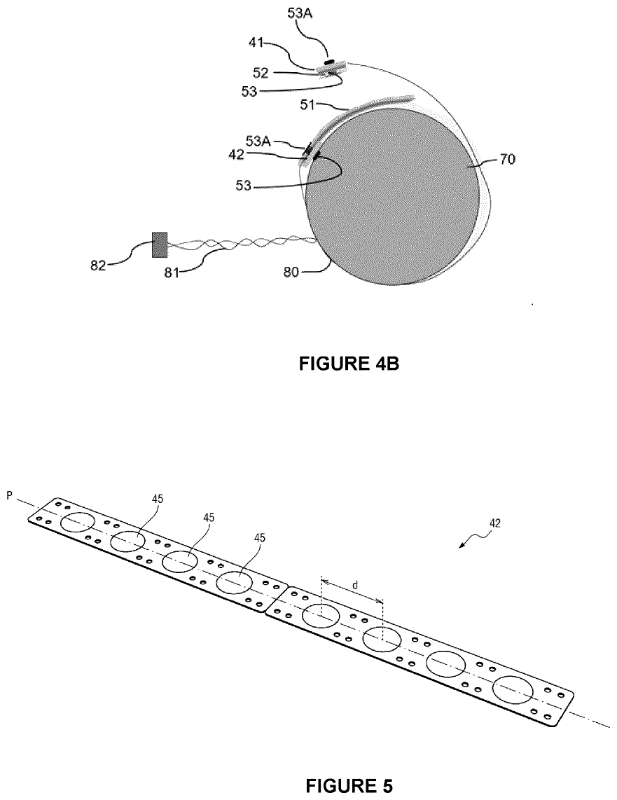

With reference to FIG. 4A, each printed circuit board 41, 42 includes at least one electrically conductive connection element 51, 52. Each connection element 51, 52 is arranged on a contact area 45 of a base of the printed circuit board and in mechanical and electrical contact with the contact area 45. For example, the connection element 51 can be soldered to the rigid printed circuit board 41, and the connection element 52 can be riveted to the flexible printed circuit board 42. The connection elements 51, 52 can also be mechanically secured to the printed circuit boards 41, 42 by another technique, for example gluing, or by solders also in the case of a flexible printed circuit board. The electrical contact between the connection element 51, 52 and the contact area 45 of the printed circuit board can be held by riveting ensuring good contact between the electrically conductive elements, or for example by soldering, gluing or sewing. Advantageously, the connection elements 52 are riveted to the flexible bases of the printed circuit board 42, ensuring a good mechanical connection and contact between the electrically conductive surfaces. In addition, the electrical contact between the connection elements 52 and the contact areas 45 can be ensured by soldering or by applying an electrically conductive glue.

With reference to FIG. 4A, when the plethysmography device 10 is worn by an individual, the textile support 20 and the inductive coils 80 surround the trunk of said individual. It is necessary to adjust the circumference of the device so that the coils 80 are kept close to the trunk, and the wavelets are in their elasticity area, preferably under slight tension. Such an arrangement of the coils allows to follow variations in the volume of the trunk that is to be detected by the device, for example a rapid variation in volume due to the cardio-respiratory activity of the individual.

While the device 10 is being worn, the connection elements 51, 52 on the bases of the printed circuit board 41, 42 at both ends of each coil 80 are mechanically engaged to ensure proper closing of the device 10 around the trunk of the mammal. Thus, the single connection element 51 arranged on the rigid base 41 is typically engaged with one of the connection elements 52 arranged on the flexible strip arranged at the opposite end of the same coil 80. The fact that one of the bases of the printed circuit board is made of a rigid material facilitates the engagement and separation of the connection elements. In particular, it facilitates handling and improves the distribution of forces if the connection elements are mechanically engaged, thus avoiding the tearing of the connection elements when they are separated. At the same time, the connection elements 51, 52 arranged on the two respective ends of each coil are electrically engaged, thereby closing the electrical circuit of each coil 80.

When the strip of the printed circuit boards 42 on a lateral edge 22 comprise a plurality of connection elements 52 spaced apart by a distance d, the plethysmography device 10 can be closed in a plurality of positions, that is to say the connection element 51 can be selectively connected to one of the connection elements 52 arranged on the printed circuit board comprising a plurality of contact areas 45. This allows to electrically close the coil 80 in a plurality of positions, each position corresponding to a different circumference of the coil 80. Thus, the circumference of the coil 80 can be chosen according to the volume of the trunk of the mammal carrying the device 10. When the weight and volume of the mammal has increased or decreased significantly, the perimeter of the coil 80 can be adjusted by separating the connection elements 51, 52 and connecting the connection element 51 to another connection element 52 on the printed circuit board comprising a plurality of contact areas 45.

With reference to FIG. 4B, the connection elements may be a male or female element 51 of a self-gripping strip and a female or male element 52 of a self-gripping strip. When wearing the device, the self-gripping strip elements are electrically and mechanically engaged along the length of the self-gripping strip element 52. The electrical connection between the printed circuit board and the self-gripping strip may be achieved by a rivet 53A, 53 and/or an electrically conductive glue.

For example, with reference to FIG. 5, the single connection element 51 of the first end of the coil 80 can be connected to any one of the connection elements 52A, 52B, 52C arranged on the printed circuit board 42 of the second end of the same coil 80. When the connection element 51 of the first end is connected to the element 52A closest to the conductive wire of the second end, the circumference of the coil 80 is the smallest. When this circumference is less than the circumference of the trunk 70 of the mammal, the single connection element 51 of the first end can be connected to a second connection element 52B of the second end and thus adapt the size of the device. In a third step, the connection element 41 of the first end can be connected with the third connection element 53C of the second end to further increase the circumference of the coil 80. The number of connection elements 51, 52 of the two ends can be different from the examples presented. Typically, the number of connection elements is comprised between 1 and 10.

When the circumference of all the coils 80 comprised in the plethysmography device 10 is adjusted by connecting the connection elements corresponding to the diameter of the trunk 70, the device 10 is thus adapted to the volume of the trunk 70 of the individual. In the same way, the circumference can be adjusted when each of the two ends of a coil 80 includes several connection elements 51, 52.

The distance d between the connection elements 52 is chosen so that the possible circumferences are sufficiently close for reliable and accurate measurements to be possible regardless of the circumference of the trunk 70. The intermediate circumferences of the trunk are taken into account by the elasticity of the fabric and the wavelet arrangement of the strand of the electrically conductive wire 81.

In other embodiments, each printed circuit board is replaced by an electrically conductive plate.

### Connection by Snap Fasteners

In a first embodiment, the connection elements 51, 52 are male elements and female elements of snap fasteners. In this case, the female elements are typically arranged on the printed circuit boards 41 on a rigid base near the first lateral edge 21 of the textile support 20, and the male elements on the strip of the printed circuit boards 42 on a flexible support near the other lateral edge 22 of the textile support 20 opposite the first lateral edge 21. In order to ensure good electrical contact between the connection elements 51, 52, the snap fasteners are made of steel with a coating of an electrically conductive material, or of an electrically conductive material, for example nickel, copper, brass or another electrically conductive alloy.

Each male or female element of a snap fastener is riveted to a contact area 45 on the base of the printed circuit board by means of a rivet head 53. The rivet head 53 is inserted into a perforation of the base from the face opposite the face including the contact area 45 and can pass through a central orifice of the male element of the snap fastener.

Advantageously, in order to ensure proper hold of the riveting and to prevent the snap fasteners from being torn off when they are opened, a rigid plate 54 is added between the head of the rivet 53 of the snap fastener and the flexible PCB base. The addition of such a plate 54 facilitates the connection, disconnection and gripping of the snap fasteners. The rigid plate 54 is typically circular and includes a perforation placed directly under the perforation of the base of the printed circuit board to insert the head of the rivet 53 simultaneously into the two perforations. Since the rigid plates are independent of each other, a certain flexibility of the device is thus preserved.

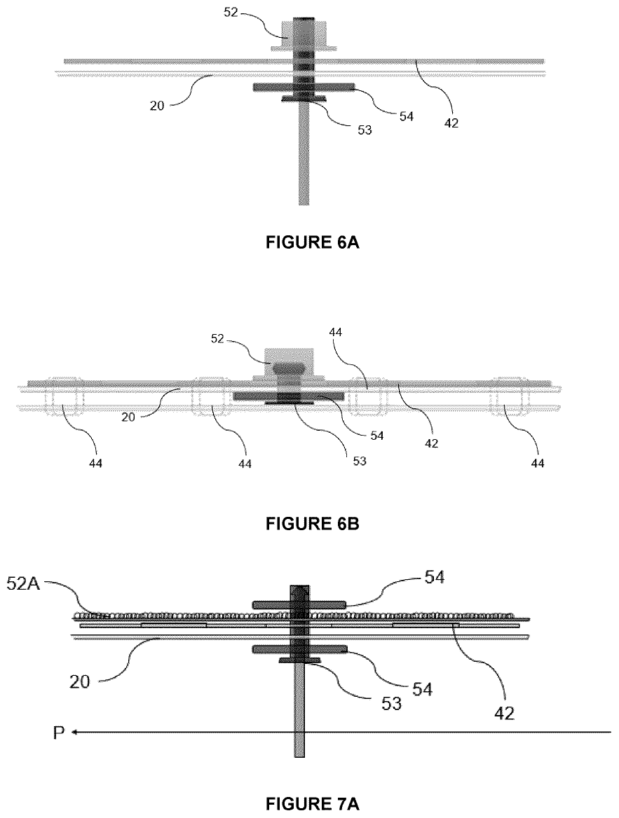

FIG. 6A illustrates the arrangement of a male or female element 52 of a snap fastener before riveting. A rivet head 53 is inserted, for example from below, into a rigid plate 54 which is for example made of a rigid plastic. The base of the printed circuit board 42 is then pierced or the rivet head 53 is inserted into a perforation already in place. Above the PCB base 42, the male or female element 52 of the snap fastener is arranged concentrically above the rivet head 53 which will subsequently be connected to the male or female element 52 by riveting. Such riveting is carried out on the flexible printed circuit board 42.

FIG. 6B illustrates the securing of a male or female element 52 of a snap fastener to a base of the printed circuit board 42 after riveting. The rigid plates 54 are firmly held against the base 42 by the rivet head 53 and the male or female element 52 of the snap fastener. Electrically conductive soldering or gluing can provide a good electrical connection between the male or female element 52 of the snap fastener and the contact area 45 arranged on the base of the printed circuit board 42.

The centers of the snap fasteners are spaced apart by a distance d which is typically the same distance between all adjacent snap fastener centers. The distance d can be adjusted to vary the range of perimeters of the trunk 70 that can be surrounded by the coils 80.

The base of the printed circuit board 42 is secured to the textile support 20 by stitching 44 along the long edges of the base 42. The dimensions of the base of the printed circuit board 42, the rigid plates, and the snap fasteners are chosen to leave sufficient space between each snap fastener and each edge of the base 42 to at least pass a presser foot of a sewing machine around the snap fastener to sew the base of the printed circuit board to the textile support in the areas arranged between the snap fasteners, so that the sewing thread can pass close to the snap fastener and does not need to be cut.

### Connection by Self-Gripping Strips

In a second embodiment, the connection elements 51, 52 are male and female hooking elements of an electrically conductive self-gripping strip system (Velcro™ type), for example made of a textile or a polymer including metal threads or particles or a metal coating.

Such a male hooking element comprises a face including hooks, and a female hooking element comprises a velvet-like face comprising loops into which the hooks of the male element can engage.

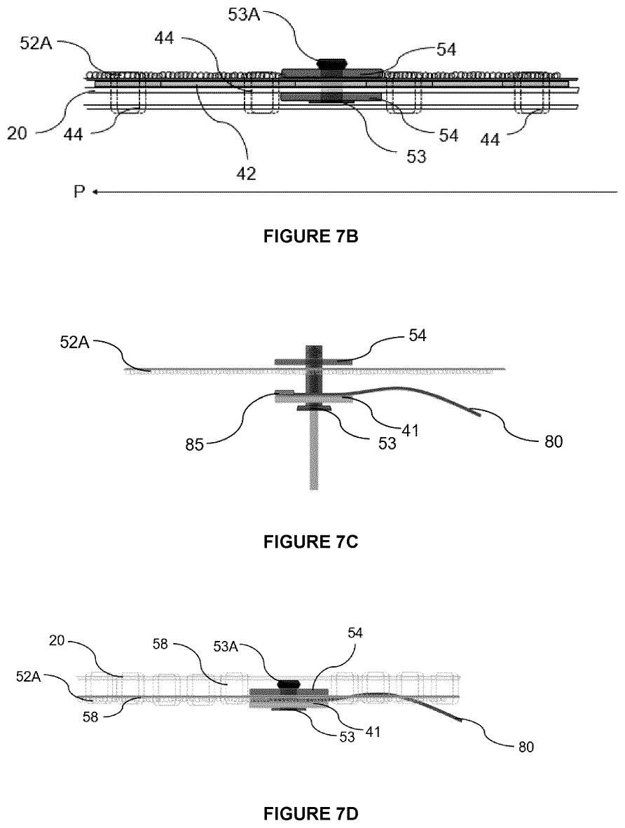

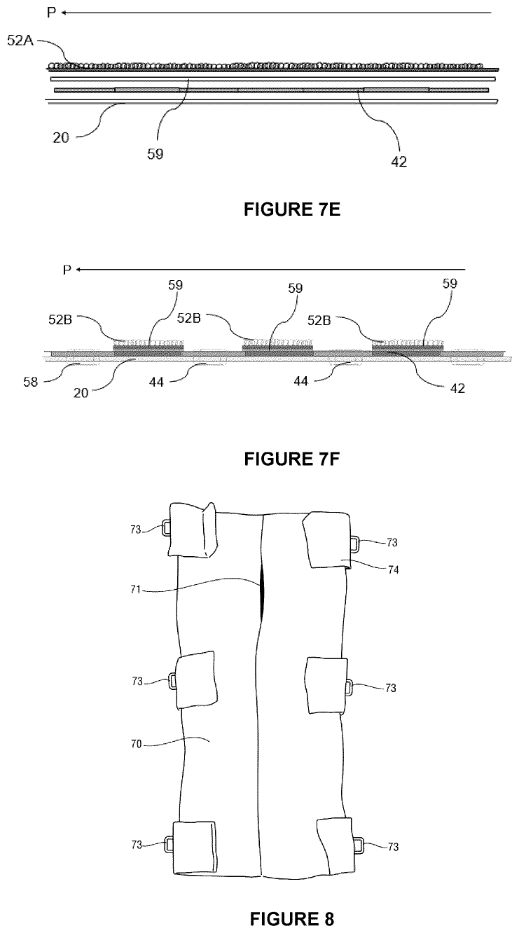

In this case, with reference to FIGS. 7A to 7C, the connection element arranged on the strip of the printed circuit board may be a single elongated self-gripping strip 52A, extending along the axis P of the strip of the printed circuit board. Thus, the circumference of the respective coil is continuously adjustable by hooking the male part and the female part according to the desired circumference. Alternatively, with reference to FIG. 7D, the connection element may be a plurality of strips 52B aligned along the axis P.

In the case where the connection elements 51, 52 are self-gripping strips, the first printed circuit board and the second printed circuit board preferably have rigid bases to facilitate the opening and closing of the self-gripping strips and to facilitate the securing of the printed circuit board on each self-gripping strip. In certain embodiments, the printed circuit board is directly produced on a rigid base. In other embodiments, the printed circuit board is produced on a flexible substrate which is reinforced by a rigid base, for example by gluing. Alternatively, the first and second electrical connection elements are electrically conductive rigid plates.

The self-gripping strips 52A, 52B may be secured to the electrical connection elements and/or the fabric support by riveting between pairs of rigid plates 54. Preferably, the rivet used for this assembly is electrically conductive.

As illustrated in FIG. 7A and FIG. 7B, the rivets and rigid plates may be secured to the entire fabric support 20 and the self-gripping strip 52A. The self-gripping strips, rigid plates, and fabric are held between the ends 53, 53A of the rivet. Alternatively, the rivets and rigid plates 54 may be secured only to the self-gripping strip 52A. In this case, the fabric support 20 is secured by a seam 58 to the self-gripping strip including the printed circuit board held by the rigid plates and a rivet.

In some embodiments, the first electrical connection element is a printed circuit board 41 arranged on a rigid base or an electrically conductive plate. The self-gripping strip 52A and/or the fabric support is riveted between the rigid base of the printed circuit board or the electrically conductive plate 41 and a single rigid plate 54, as illustrated in FIG. 7C and FIG. 7D. The fabric support 20 may be comprised between the rigid plate 54 and the rigid base of the printed circuit board 41 as illustrated in FIG. 7C. Alternatively, as illustrated in FIG. 7D, the fabric support 20 may be secured to the self-gripping strip only by stitching 58, thereby covering the printed circuit board 41.

The coil 80 is secured by a solder 85 on the printed circuit board or the electrically conductive plate 41. Preferably, the solder 85 is arranged between the rigid base and the self-gripping strip 52A in order to hold the solder 85 by riveting between the rigid base of the printed circuit board or the electrically conductive plate 41 and the rigid plate 54. This allows good hold of the solder during the opening and closing of the self-gripping strips.

Such riveting allows to very easily and very simply connect the end of each coil to each self-gripping strip, ensuring mechanical contact and good mechanical strength of the connection.

Preferably, at least the rigid plate in contact with the self-gripping strip is electrically conductive. Thus, together with an electrically conductive rivet, electrical contact can be ensured between the self-gripping strip 52A and the printed circuit board or the electrically conductive plate. It is therefore not necessary for the self-gripping strip to be in contact with the printed circuit board or the electrically conductive plate to ensure the electrical connection between the self-gripping strip and the printed circuit board or the electrically conductive plate.

Advantageously, the printed circuit board or electrically conductive plate is connected to the self-gripping strip by a single rivet. Such an arrangement simplifies the assembly of the printed circuit board or electrically conductive plate, while allowing continuous adjustment by means of the self-gripping strip.

Alternatively, the use of an elongated printed circuit board allows a plurality of electrical connections to be made with the self-gripping strips so as to shorten the path traveled by the electric current in the self-gripping strips. Since the electrical resistivity of the self-gripping strips is generally higher than that of the printed circuit board, such a plurality of electrical connections allows the total impedance of the circuit to be reduced.

In some embodiments, the gripping strips 52A, 52B may be glued to the printed circuit board or electrically conductive plate by an electrically conductive glue 59 as illustrated in FIG. 7E and FIG. 7F. The gluing may be performed in one or more areas or over the entire surface of the strip.

The mechanical and electrical connection may be provided only by electrically conductive glue or by electrically conductive glue and one or more rivets. In the latter case, the rivets may be electrically non-conductive.

With reference to FIGS. 6B, 7B and 7D, the fabric, the printed circuit board or the electrically conductive plate and, where appropriate, the gripping strip can be held in place by stitching 44, preferably buttonhole type stitching.

It is possible to combine several securing modes to increase the stability and thus the service life of the plethysmography device.

### Covering Part

The plethysmography device 10 may further comprise a covering part 70 made of extensible textile. Preferably, the covering part 70 is made of the same type of textile as the textile support 20. The covering part 70 may be secured to the textile support 20 to cover the lateral edges 21, 22 of the textile support 20 and the ends of the coils 80 comprising the printed circuit boards or electrically conductive plates 41, 42 and the connection elements 51, 52. Thus, the covering part 70 protects the opening of the device 10, the printed circuit boards or electrically conductive plates 41 and the connection elements 51, 52 in order to prevent the individual wearing the device 10 from removing the device 10 or from damaging one or more of these elements.

In this case, the textile support 20 advantageously includes securing elements 47 arranged to secure the covering part 70, and the covering part 70 includes complementary securing elements 73 to be secured to the textile support 20. For example, the textile support may include hooks 47 and the covering part 70 includes loops 73 secured by means of a fabric stabilizing part 74 on the lateral edges of the covering part 70. With reference to FIG. 1, hooks 47 designed to receive such loops 73 are arranged on the external face of the textile support 20.

The covering part 70 may include a pocket including an opening 71 into which a measuring device can be inserted. Advantageously, the opening 71 of the pocket is oriented towards the trunk of the individual to prevent accidental exit of the measuring device. The measuring device may be electrically connected to the coils of the device by the electrical connector. Such a pocket allows to maintain the measuring device close to the coils 80 and to protect it so that the mammal wearing the plethysmography device 10 or fellow mammals cannot remove or damage the measuring device.

Advantageously, with reference to FIG. 8, the covering part 70 and the pocket are formed in a single part of textile, using a folding technique and stitches to form the pocket. Such a technique is easy to perform and does not require any complex cutting or dedicated tools or patterns.

1. An inductance plethysmography device, comprising:
a support made of elastic textile designed to be closed on itself along two lateral edges in order to surround at least a portion of a trunk of an individual,
at least one inductive coil formed of an electrically conductive wire strand arranged in a form of wavelets between the two lateral edges so as to extend along a circumference of the individual's trunk, the at least one inductive coil having two ends arranged on each lateral edge of the support,
wherein a first end of the at least one inductive coil comprises a first electrical connection element electrically connected to the electrically conductive wire strand, said first electrical connection element being electrically and mechanically connected to an electrically conductive male or female self-gripping element,
and a second end of the at least one inductive coil comprises a second electrical connection element electrically connected to the electrically conductive wire strand, said second electrical connection element being electrically and mechanically connected to an electrically conductive female or male self-gripping element designed to electrically and mechanically engage with the electrically conductive male or female self-gripping element in order to make an electrical contact between the first and the second end of the at least one inductive coil.

2. The device according to claim 1, wherein each electrical connection element is a printed circuit board or an electrically conductive rigid plate.

3. The device according to claim 1 comprising a plurality of female or male self-gripping elements electrically and mechanically connected to the second electrical connection element, said female or male self-gripping elements being spaced apart by a distance between two successive self-gripping elements, such that each female or male self-gripping element defines a different circumference of the at least one inductive coil when said female or male self-gripping element is engaged with the male or female self-gripping element electrically and mechanically connected to the first electrical connection element.

4. The device according to claim 1, wherein the first electrical connection element and the second electrical connection element each have a respective rigid base.

5. The device according to claim 1, wherein each male or female self-gripping element is assembled to the first electrical connection element or to the second electrical connection element by an electrically conductive glue.

6. The device according to claim 1, wherein each male or female self-gripping element is assembled to the first electrical connection element or to the second electrical connection element by at least one rivet.

7. The device according to claim 6, wherein said rivet is electrically conductive.

8. The device according to claim 7, wherein each self-gripping element and each respective electrical connection element are arranged between two rigid plates connected by said rivet.

9. The device according to claim 8, wherein the first electrical connection element and the second electrical connection element each have a respective rigid base and each self-gripping element and/or the support is arranged between a rigid plate and the electrical connection element, the rigid plate and the electrical connection element being mechanically and electrically connected by said rivet.

10. The device according to claim 1, further comprising at least one pair of hooks arranged on each lateral edge of the support so as to mechanically connect said lateral edges to close the support.

11. The device according to claim 1, further comprising a removable elastic textile covering part arranged to cover the male and/or female self-gripping elements when the at least one inductive coil is closed.

12. The device according to claim 11, wherein the support comprises at least one pair of hooks arranged on each lateral edge and the removable elastic textile covering part includes at least one pair of loops arranged to be hooked onto said least one pair of hooks so as to secure the removable elastic textile covering part to the support and to cover the lateral edges of the support.

13. The device according to claim 11 wherein the removable elastic textile covering part further comprises a pocket configured to contain a measuring device electrically connected to the at least one inductive coil.

14. A method for manufacturing an inductance plethysmography device for a human or animal mammal according to claim 1, comprising
providing a support made of elastic textile,
forming at least one inductive coil from an electrically conductive wire by securing said wire in the form of wavelets along the circumference of the support, said at least one inductive coil having two ends arranged on each lateral edge of the support,
assembling each end of the at least one inductive coil with an electrical connection element, so as to establish an electrical connection between the at least one inductive coil and a conductive track of the electrical connection element,
assembling each electrical connection element with a respective male or female self-gripping element.

15. The method according to claim 14, further comprising assembling at least one electrical connection element with at least one additional self-gripping element, said self-gripping elements being spaced apart by a distance extending over a segment of a circumference of the inductance plethysmography device.

16. The method according to claim 14, comprising providing a removable elastic textile covering part comprising a plurality of loops, and assembling the support with a plurality of hooks on both sides of the opening of said support, said plurality of loops being configured to receive the plurality of hooks so as to secure the removable elastic textile covering part to the support.
